(19) United States
(12) Patent Application Publication
(10) Pub. No.: US 2019/0255195 A1
(43) Pub. Date: Aug. 22, 2019

(54) DETECTABLE MOLECULES THAT TARGET AND BIND TO MALIGNANT TUMORS AND NOT TO BENIGN TUMORS

(71) Applicant: CAO GROUP, INC., West Jordan, UT (US)

(72) Inventor: Steven D. Jensen, West Jordan, UT (US)

(21) Appl. No.: 16/345,073

(22) PCT Filed: Oct. 25, 2017
(86) PCT No.: PCT/US17/58276

§ 371 Date: Apr. 25, 2019

Related U.S. Application Data
(60) Provisional application No. 62/412,948, filed on Oct. 26, 2016.

(51) Int. Cl. A61K 49/00; A61B 5/00

(52) U.S. Cl. CPC A61K 49/006; G01N 2800/7028; A61K 2123/00; A61B 5/0059

(57) ABSTRACT

Targeting molecules that when circulated within the bloodstream tend to bind to malignant cells and minimize attachment to healthy tissue cells and benign tumor cells. The targeting molecule is designed to be detectable such that it can be identified by various means such as an MRI, X-ray, ultrasound, chromatically visible to the eye, and any other useful means of detection.

### CROSS-REFERENCES TO RELATED APPLICATIONS

This application claims the benefit of PCT Application No. PCT/US2017/058276, filed Oct. 25, 2017, which is a non-provisional of U.S. Provisional Application No. 62/412,948 filed on Oct. 26, 2016, which is hereby incorporated by reference in its entirety.

### TECHNICAL FIELD OF THE INVENTION

The present invention discloses detectable molecules that target and bind to malignant tumors and not benign tumors, and related methods.

### SUMMARY

Embodiments of the present invention provide detectable molecules that target and bind to malignant tumors and not benign tumors, and related methods.

### DETAILED DESCRIPTION OF THE INVENTION

The contemporary means to determine if a tumor is malignant or benign is usually a biopsy followed by a histological examination. A biopsy of a malignant tumor is a high-risk surgical operation as it cuts into the tumor and dislodges cancerous cells that can be circulated throughout the body via the blood stream (metastasize). A biopsy of a malignant tumor is a means to increase the spread of cancer throughout the body.

What is needed are means to determine if a tumor is malignant or benign without a biopsy. The present invention utilizes targeting molecules that when circulated within the bloodstream tend to bind to malignant cells and minimize attachment to healthy tissue cells and benign tumor cells. The targeting molecule is designed to be detectable such that it can be identified by various means such as an MRI, X-ray, ultrasound, chromatically visible to the eye, and any other useful means of detection.

A medical professional by practice of the present invention would introduce the targeting molecule into the bloodstream of the patient and allow the molecule to circulate and bind or not to bind to the tumor(s) in question. After a prescribed interval, the practitioner would examine the tumor site with respect to the detection properties of the targeting molecule; this is done by various means such as an MRI, X-ray, ultrasound, chromatically visible to the eye, and any other useful means of detection. For example, if the targeting molecule was radiopaque, then an X-ray radiograph would be utilized to determine if the targeting molecule accumulated within the tumor in question or not. The detection of a malignant tumor would be determined if the tumor was radiographically anomalous.

The present invention utilizes peptides, polypeptides, polymers, proteins, biopolymers and phospholipids as targeting molecules that are known to collect and bind within cancer cells. An embodiment of the present invention prefers targeting molecules that bind to fibrin and fibrinogen. Another embodiment of the present invention prefers targeting molecules that have an affinity to bind to pleomorphic cells. Another embodiment of the present invention prefers targeting molecules that have an affinity to bind to neoplastic cells. Another embodiment of the present invention prefers targeting molecules that bind to both malignant and benign tumors, yet takes advantage of the differences in cell densities within these tumors such that there is a measurable difference between malignant and benign tumors that can be quantified and measured. Another embodiment of the present invention prefers targeting molecules that bind to DNA. Another embodiment of the present invention prefers targeting molecules that bind to the nucleus.

A list of peptides, polypeptides, polymers, biopolymers and proteins that have an affinity to bind fibrinogen and fibrin are found in U.S. Pat. No. 8,513,380 and is hereby incorporated in its entirety by reference. U.S. Pat. No. 8,513,380 also disclose the means of manufacture and the means to discover additional peptides when applied in practice.

Phospholipids that have an affinity to bind to cancer cells are found in U.S. Pat. No. 4,935,520 and is hereby incorporated in its entirety by reference. U.S. Pat. No. 4,935,520 also disclose the means of manufacture and the means to discover additional peptides when applied in practice.

The present invention is designed to target the inherent differences between malignant and benign cells within a tumor such as: cell density, cellular and nuclear pleomorphism, the nuclear to cytoplasmic ratio, DNA density, nuclear density, and any other useful differentiation between cells and tumors alike.

An embodiment of the present invention selects targeting molecules that have a greater affinity to bind to malignant tumors and at the same time minimizes binding to benign tumors and healthy tissue; to these are added chemical moieties that allow them to be detected by various means such as an MRI, X-ray, ultrasound, chromatically visible to the eye, and any other useful means of detection.

A radio-opaque targeting molecule is created by adding a radio-opaque moiety onto a peptide, polypeptides, polymers, proteins, biopolymers and phospholipids. The present invention utilizes heavy elements as a source of radio-opaque substances such as: iodine, bromine, calcium, barium, strontium, bismuth, tungsten, zirconium, iron, copper, nickel, zinc, silver, tin, gallium, antimony, palladium, rhodium, yttrium, molybdenum, cobalt, chromium, titanium, vanadium, magnesium, gold, platinum, and iridium and any other radiographically visible substance. These radio-opaque substances can be used in their elemental form, as a salt, bound in chelated form, or as an organometallic compound. Any radio-opaque moiety that can be attached to a targeting molecule that does not inhibit the resultant compound's ability to collect and bind to cancer cells is within the scope of this patent.

A MRI detectable targeting molecule is created by adding a MRI contrasting moiety onto a peptide, polypeptides, polymers, proteins, biopolymers and phospholipids. The present invention utilizes gadolinium, iron oxides, iron platinum, manganese, and any other MRI contrastable agent as MRI contrasting moieties. These MRI contrasting moieties can be used in their elemental form, as a salt, bound in chelated form, or as an organometallic compound. Any MRI contrasting moiety that can be attached to a targeting molecule that does not inhibit the resultant compound's ability to collect and bind to cancer cells is within the scope of this patent.

A chromatic targeting molecule is created by adding a chromatic moiety onto a peptide, polypeptides, polymers, proteins, biopolymers and phospholipids. The present invention utilizes: conjugated organic compounds, organometallic compounds and any other useful compounds of color. Any chromatic moiety that can be attached to a targeting molecule that does not inhibit the resultant compound's ability to collect and bind to cancer cells is within the scope of this patent.

A targeting molecule that is detectable by ultrasound is created by adding a hollow nano-sphere moiety onto a peptide, polypeptides, polymers, proteins, biopolymers and phospholipids.

What is claimed is:

1. Detectable molecules that target and bind to malignant tumors and not benign tumors.
